(19) United States
(12) Patent Application Publication
(10) Pub. No.: US 2008/0014156 A1
(43) Pub. Date: Jan. 17, 2008

(54) PHOTOPROTECTANT COMPOSITION FOR PREVENTING SUNBURN AND SUN DAMAGE TO THE SKIN

(76) Inventor: Gregory T. Horn, Lighthouse Point, FL (US)

Correspondence Address: EDWARDS ANGELL PALMER & DODGE LLP, P.O. BOX 55874, BOSTON, MA, 02205, US

(21) Appl. No.: 11/776,874

(22) Filed: Jul. 12, 2007

Related U.S. Application Data
(60) Provisional application No. 60/830,439, filed on Jul. 12, 2006.

(51) Int. Cl. A61K 8/97; A61K 36/11; A61Q 17/04

(52) U.S. Cl. 424/59; 424/763

(57) ABSTRACT

Photoprotectant compositions and methods of using the compositions to protect skin from sunburn and sun damage are disclosed.

### CROSS-REFERENCE TO RELATED APPLICATIONS

This application claims the benefit of priority to U.S. Provisional Patent Application No. 60/830,439, filed Jul. 12, 2006, the contents of which are incorporated by reference herein in their entirety.

### BACKGROUND OF THE INVENTION

Sun exposure, and particularly exposure to ultraviolet wavelengths of sunlight, causes damage to the skin, resulting in photoaging (cosmetic damage to skin) as well as medical conditions such as sunburn, and, in some cases, skin cancers. Current methods for protecting against damage from sunlight include chemical sunscreens. While these compounds can be effective in reducing exposure to damaging sunlight, some compounds are suspected of acting as endocrine disruptors when absorbed through the skin. Also, chemical sunscreens generally do not protect against all forms of photoaging caused by sun exposure. Cost and convenience, including the need for frequent topical re-application, further limit the effectiveness of chemical topical sunscreens. Physical sunscreens, such as zinc oxide and titanium dioxide, also can be effective sunblocks, but topical application and cosmetic appearance of these sunblocks can limit their acceptance.

Certain oral supplements have been reported to provide protection from sunburn. For example, astaxanthin, a carotenoid pigment, has been reportedly used for sun protection, see, e.g., U.S. Pat. No. 6,433,025; the mechanism of action is believed to be at least in part due to the antioxidant properties of astaxanthin. Extracts of ferns of the genus Polypodium have been reported to be photoprotective, as discussed in U.S. Pat. No. 5,614,197, possibly due to an immunomodulatory effect. The use of antioxidants and other nutritional substances to retard photoaging and its mechanisms (primarily through MMP (matrix metalloproteinase) inhibition for sun protection are discussed in U.S. Patent Application 20050058709.

Additional compositions and methods for the prevention of photoaging, sunburn, and other forms of damage resulting from exposure to ultraviolet radiation would be desirable.

### SUMMARY

The invention relates generally to photoprotectant compositions and to methods of using the compositions to protect skin from sunburn and sun damage.

In one aspect, the invention provides a photoprotectant composition including at least one carotenoid and a Polypodium extract.

In certain embodiments, the at least one carotenoid comprises a carotenoid selected from the group consisting of astaxanthin, lycopene, canthaxanthin, β-carotene, lutein, and zeaxanthin, more preferably astaxanthin and/or lycopene. In certain embodiments, the Polypodium extract comprises an extract of a plant selected from the group consisting of Polypodium aureum, Polypodium crassifolium, Polypodium decumanum, Polypodium lanceolatum, Polypodium leucotomos, Polypodium percussum, Polypodium triseriale and Polypodium vulgare, more preferably Polypodium leucotomos extract.

In another aspect, the invention provides a composition prepared by combining at least one carotenoid with a Polypodium extract. In certain embodiments, the Polypodium extract is an extract of Polypodium leucotomos.

In another aspect, the invention provides a pharmaceutical composition including an effective photoprotectant amount of at least one carotenoid and a Polypodium extract, together with a pharmaceutically-acceptable carrier.

In preferred embodiments of the pharmaceutical composition, the at least one carotenoid is a carotenoid selected from the group consisting of astaxanthin, lycopene, canthaxanthin, β-carotene, lutein, and zeaxanthin, more preferably astaxanthin and/or lycopene. In certain embodiments, the Polypodium extract comprises an extract of a plant selected from the group consisting of Polypodium aureum, Polypodium crassifolium, Polypodium decumanum, Polypodium lanceolatum, Polypodium leucotomos, Polypodium percussum, Polypodium triseriale and Polypodium vulgare, more preferably Polypodium leucotomos extract. In certain embodiments, the pharmaceutical formulation is formulated for oral administration, more preferably in the form of a tablet, such as a chewable tablet; or a two-piece capsule, such as a soft gelatin capsule. In certain embodiments, the pharmaceutical formulation is in the form of a liquid. In certain embodiments, the pharmaceutical formulation is a controlled-release formulation. In certain embodiments, the pharmaceutical formulation is a sustained-release formulation.

In certain embodiments, the pharmaceutical formulation also includes an anti-oxidant such as superoxide dismutase, N-acetyl cysteine or selenium.

In another aspect, the invention provides a packaged product including a container, a pharmaceutical composition of the invention including an effective photoprotectant amount of at least one carotenoid and a Polypodium extract, together with a pharmaceutically-acceptable carrier, and instructions for oral administration of the pharmaceutical composition to provide photoprotection.

In another aspect, the invention provides a method of providing photoprotection to an individual, the method comprising administering to the individual a pharmaceutical composition comprising an effective photoprotectant amount of at least one carotenoid and a Polypodium extract.

In certain embodiments of the inventive methods, the step of administering comprises administering the pharmaceutical composition before, during, or after sun exposure, preferably two to three days before sun exposure.

In another aspect, the invention provides a pharmaceutical composition in unit dose form. The pharmaceutical composition includes about 1 mg to about 6 mg astaxanthin; about 1 mg to about 25 mg lycopene; about 1 mg to about 300 mg Polypodium extract; and a pharmaceutically acceptable carrier.

Other embodiments of the invention will be apparent from the specification and claims which follow.

### DETAILED DESCRIPTION OF THE INVENTION

The term “carotenoid,” as used herein, is art-recognized and refers to a member of a family of organic pigments characterized by a polyene chain. Examples of carotenoids include carotenes, such as lycopene, α-carotene and β-carotene, and xanthophylls, such as canthaxanthin, astaxanthin, lutein and zeaxanthin.

The term “Polypodium extract” extract, as used herein, refers to an extract of a plant of the genus Polypodium. Polypodium species include Polypodium aureum, Polypodium crassifolium, Polypodium decumanum, Polypodium lanceolatum, Polypodium leucotomos, Polypodium percussum, Polypodium triseriale and Polypodium vulgare. An extract of Polypodium for use in the compositions and methods of the present invention should have photoprotectant activity. Plant extracts can be described based on a reference compound found in the plant, or can refer to a concentration (e.g., weight/weight) of the total extract compared to the whole or dried plant material. An extract can be, e.g., a 30:1 or 50:1 extract.

The terms “photoprotective” and “photoprotectant”, as used herein, refer to the property of protecting skin from damage due to exposure of the skin to ultraviolet (UV) radiation (e.g., sunlight), including UVA (long-wave UV), UVB (shorter-wave UV), or both UVA and UVB, or to a composition having such a property. Compounds and compositions of the invention are generally capable of achieving one or more of the following effects: the inhibition or retardation of skin inflammation, erythema or sunburn reaction and tissue damage to skin and/or the inhibition or retardation of the immediate pigment darkening reaction and/or the inhibition or retardation of the delayed tanning reaction and/or the inhibition or retardation of phototoxic reaction produced by psoralens. A compound or composition providing such photoprotection is said to be “photoprotective” and may be referred to as a “photoprotectant.”

While the description herein generally refers to sunburn or sun damage, it will be appreciated that the invention also related to compositions, kits and methods for preventing, treating, or ameliorating damage to skin caused by exposure to ultraviolet radiation from sources other then the sun (e.g., from tanning beds and the like)

Compositions

In one aspect, the invention provides a photoprotectant composition including at least one carotenoid and a Polypodium extract.

Examples of carotenoids include carotenes, such as lycopene, α-carotene and β-carotene, and xanthophylls, such as canthaxanthin, astaxanthin, lutein and zeaxanthin.

Astaxanthin is one of the most powerful naturally-occurring carotenoid antioxidants (up to 550 times more effective than vitamin E). Astaxanthin is believed to protect cells against oxidation by scavenging free radicals. Astaxanthin has been shown to protect the skin from the damaging effects of ultraviolet radiation (UVR), thereby allowing for longer exposure to sunlight without sunburn, lessening of chronic sun blisters and those with sensitivity to sunlight attained significantly higher tolerance to exposure. Astaxanthin can be obtained from naturally-occurring sources (e.g., from marine algae) or can be synthetically produced; astaxanthin from naturally-occurring sources is preferred in this invention. Synthetic astaxanthin can be purchased from commercial sources; similarly, astaxanthin from natural sources can be purchased, e.g., from Cyanotech Corporation. Other synthetic or natural carotenoids, including lycopene, can also be purchased from commercial sources.

Lycopene is a naturally-occurring carotenoid with powerful antioxidant properties. During UV irradiation, skin is exposed to photooxidative damage, resulting in premature aging of the skin, photodermatoses and skin cancer. Lycopene has been shown to be an efficient free radical scavenger and to prevent UV light-induced skin lesions, and may protect against skin cancer. Lycopene can be obtained from naturally-occurring sources (e.g., from tomato) or can be synthetically produced; lycopene from naturally-occurring sources is preferred in this invention. Lycopene preparations can be purchased, e.g., from LycoRed Corporation).

In a preferred embodiment, the invention provides compositions which include a combination of two (or more) carotenoids. Particularly preferred combinations include the combination of astaxanthin with lycopene.

In general, the composition will contain an effective photoprotectant amount of the at least one carotenoid when combined with the Polypodium extract. Thus, in certain embodiments, the at least one carotenoid will be present in an amount sufficient to provide a daily dosage of 1-50 mg of carotenoid (either as an individual carotenoid or a combination of two or more carotenoids). In certain embodiments, the at least one carotenoid is a combination of astaxanthin and lycopene in a total amount of between 1 and 50 mg, e.g., an amount effective to deliver a daily amount of 1-6 mg astaxanthin/day and 1-50 mg lycopene/day.

Calaguala fern (polypodium leucotomos) is a natural botanical with powerful sun protecting properties. Polypodium leucotomos (extract) helps maintain the skin's tolerance to the sun, protects skin elastin, protects the epidermal immune system by preserving the cells of the Lagerhans, and protects DNA by inhibiting the formation of thymine dimers. Polypodium is preferably provided in the form of an extract of the fern. In general, a Polypodium extract ranging from 10:1 to 50:1 will be provided in an amount of 1 mg-300 mg per day. Polypodium extracts can be purchased from commercial sources, e.g., from Organic Hope Corporation.

The combination of a combination of carotenoids, e.g., astaxanthin with lycopene, with a Polypodium extract provides a broader spectrum of antioxidant protection from the carotenoids (e.g., astaxanthin and lycopene) than either can provide alone, along with the complimentary benefit of modulation of the skin's immune response to sun damage from Polypodium. This novel multi-action approach provides protection both from the direct damage caused by UV exposure as well protecting the body's immune response to that damage. The invention thus provides dual-acting complimentary ingredients to provide both broad-spectrum antioxidant and immunomodulating protection from UV damage.

Pharmaceutical Compositions

The pharmaceutical compositions of the present invention comprise a therapeutically effective amount of (i) at least one carotenoid and (ii) a Polypodium extract, formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally. Thus, in a preferred embodiment, the invention provides pharmaceutical formulations for oral administration.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In certain embodiments, the dosage form is a two-piece hardshell gelatin capsule; a two-piece hardshell vegetable gelatin capsule; a soft gelatin capsule; a vegetarian soft gelatin capsule; or a tablet. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

In certain embodiments, the invention provides topical formulations. In addition to the at least one carotenoid and the Polypodium extract, a topical preparation may include physical sunscreen agents, chemical sunscreen agents and/or cosmetic agents. In particular, a physical sunscreen agent such as titanium dioxide, silicone-treated titanium dioxide, zinc oxide, ferrous oxide, ferric chloride, talc, chromium oxide, or cobalt oxides may be included. Alternatively or in addition, a chemical sunscreen agent such as para-amino benzoic acid, esters of para-amino benzoic acid, salicylates, cinnamates, benzophenones, dihydroxyacetone, parsol 1789, or melanin may be included.

Methods and Administration

In one aspect, the invention provides a method of providing photoprotection to an individual, such as a human. The method includes the step of administering to the individual a pharmaceutical composition comprising an effective photoprotectant amount of at least one carotenoid and a Polypodium extract.

In another aspect, the invention provides a method of preventing sunburn or skin damage caused by exposure to ultraviolet radiation. The method includes the step of administering to an individual in need thereof, such as a human, a pharmaceutical composition comprising an effective photoprotectant amount of at least one carotenoid and a Polypodium extract.

In the above-referenced methods, in certain embodiments, the at least one carotenoid comprises a carotenoid selected from the group consisting of astaxanthin, lycopene, canthaxanthin, β-carotene, lutein, and zeaxanthin. In certain embodiments, the at least one carotenoid comprises astaxanthin. In certain embodiments, the at least one carotenoid comprises lycopene. In certain embodiments, the at least one carotenoid comprises astaxanthin and lycopene.

In certain embodiments, the polypodium extract comprises an extract of a plant selected from the group consisting of Polypodium aureum, Polypodium crassifolium, Polypodium decumanum, Polypodium lanceolatum, Polypodium leucotomos, Polypodium percussum, Polypodium triseriale and Polypodium vulgare. In certain embodiments, the polypodium extract comprises Polypodium leucotomos extract.

In certain embodiments, the pharmaceutical composition or formulation is formulated for oral administration. In certain embodiments, the pharmaceutical formulation is in the form of a tablet. In certain embodiments, the pharmaceutical formulation is in the form of a two-piece capsule. In certain embodiments, the pharmaceutical formulation further comprises superoxide dismutase. In certain embodiments, the pharmaceutical formulation further comprises N-acetyl cysteine.

In certain embodiments, the step of administering comprises administering the pharmaceutical composition before, during or after sun exposure. In certain embodiments, the step of administering comprises administering the pharmaceutical composition two to three days before sun exposure.

The compositions of the invention are generally administered orally, although in certain embodiments the compositions may be topically applied. In certain embodiments, a daily dose of the composition provides about 1-50 mg of carotenoid (either as an individual carotenoid or a combination of two or more carotenoids). In certain embodiments, the at least one carotenoid is a combination of astaxanthin and lycopene in a total amount of between about 1 and about 50 mg, e.g., an amount effective to deliver a daily amount of 1-6 mg astaxanthin/day and 1-50 mg lycopene/day. In certain embodiments, a daily dose of the composition provides about 1 mg-300 mg per day of a Polypodium extract (with the extract generally being from 10:1 to 50:1 extract), more preferably about 100-150 mg of Polypodium extract.

Because Polypodium extract appears to be absorbed better under acid conditions, the capsules are preferably not taken with an antacid and, most preferably, are taken at least one half hour before a meal. Similarly, because alcohol appears to reduce the absorption and efficacy of Polypodium extract, the preparation is preferably not administered with alcoholic beverages.

As Polypodium extract appears to enhance the effect of the drug digitalis, individuals Taking digitalis should consult a physician before using an orally administered Polypodium extract preparation and, if appropriate, the dosage of digitalis should be reduced. Preferably, Polypodium extract is not used by individuals taking digitalis.

In general, the compositions of the invention should be before, during or after sun exposure, but most preferably before sun exposure. Carotenoids are generally fat-soluble compounds, and, in some cases, it may be desirable to increase the level of carotenoid present in the body by administering a carotenoid-containing composition of the invention over an extended period of time. In certain embodiments, dosages of the compositions of the invention should be taken beginning two to three days before sun exposure, or a week before sun exposure. However, Polypodium extracts are generally more water-soluble and can therefore be effective even when administered in a relatively short time period before sun exposure. In certain embodiments, dosages of the compositions of the invention should be taken beginning 24 hrs prior to solar or other ultraviolet radiation exposure, or within 12 hours prior to exposure. In addition, daily dosages may be taken in the days preceding exposure. In certain embodiments, dosages of the compositions of the invention should be taken within six hours or less of sun exposure. In certain embodiments, dosages of the compositions of the invention should be taken within three hours or less of sun exposure. In certain embodiments, dosages of the compositions of the invention should be taken within one hour or less of sun exposure.

For children over 6 years of age, dosages should generally be limited to 120-240 mg of Polypodium extract per day.

The invention is illustrated by the following non-limiting embodiments.

### EXAMPLE 1

An example of a composition of the invention is a dietary supplement containing the following:

A gelatin capsule is filed with

148 mg natural astaxanthin material from algae (Cyanotech Corp.), containing about 1.35% astaxanthin;
157 mg natural lycopene from tomatoes (LycoRed Corp.), containing 5% lycopene—5% average included for shelf life;
Polypodium (preferably Polypodium leucomotos) 30:1 extract 200 mg (Organic Hope Corp.)
   Excipients and carriers used in the manufacturing process including vegetable stearates, silicon dioxide, and croscarmellose sodium.

In this formulation, one capsule per day provides approximately 2 mg of astaxanthin, 7.5 mg of lycopene from tomatoes, and 200 mg of polypodium extract.

Other formulations include astaxanthin, lycopene and polypodium 30:1 extract in any ratio of 2.0 mg:7.5 mg:200 mg.

### EXAMPLE 2

An example of a composition of the invention is a dietary supplement containing the following:

A chewable tablet is made by combining

79 mg natural astaxanthin material from algae (Cyanotech Corp.), containing about 1.35% astaxanthin;
157 mg natural lycopene from tomatoes (LycoRed Corp.), containing 5% lycopene—5% average included for shelf life;
Polypodium (preferably Polypodium leucomotos) 30:1 extract 150 mg (Organic Hope Corp.)

Exemplary excipients, binders, and carriers that can be used in the manufacturing process including vegetable stearates, silica, and maltodextrin.

Flavoring and sweetening agents such as xylitol, mannitol, sorbitol, lo han fruit, fructose, and natural flavors can also be used in the compositions of the invention.

In this formulation, two tablets per day provide approximately 2 mg of astaxanthin, 15 mg of lycopene from tomatoes, and 300 mg of polypodium extract.

### EXAMPLE 3

Pills are prepared such that two pills per day provide:

Astaxanthin (preferably natural source) 4.0 mg
(from 296 mg natural astaxanthin material from algae, containing 1.35% astaxanthin, Cyanotech Corp.)

Lycopene (preferably natural source) 15.0 mg
(from 315 mg natural lycopene from tomatoes, containing 5% lycopene—5% average included for shelf life, LycoRed Corp.)

Polypodium (preferably polypodium leucomotos) 30:1 extract 400 mg (Organic Hope Corp.).

In certain embodiments, one or two pills per day provide:

Superoxide Dismutase (preferably from stabilized SOD) 50 mg (PL Thomas and Co. Inc.)

Selenium (as selenomethionine) 200 mcg (Pharmachem Corp.)

Astaxanthin (preferably natural source) 2.0 mg
(from 148 mg natural astaxanthin material from algae, containing 1.35% astaxanthin, Cyanotech Corp.)

Lycopene (preferably natural source) 7.5 mg
(from 157 mg natural lycopene from tomatoes, containing 5% lycopene—5% average included for shelf life, from LycoRed Corp.))

Polypodium (preferably polypodium leucomotos) 30:1 extract 200 mg (from Organic Hope Corp.).

### EXAMPLE 4

Standard gelatin capsules were filled with 148 mg natural astaxanthin material from algae (Cyanotech Corporation), containing about 1.35% astaxanthin; 157 mg natural lycopene from tomatoes (commercially supplied by LycoRed Corp.), containing 5% lycopene—5% average included for shelf life; and Polypodium (preferably Polypodium leucomotos) 30:1 extract 200 mg (commercially supplied by Organic Hope Corporation), along with excipients and carriers normally used in the capsule manufacturing process including vegetable stearates, silicon dioxide, and croscarmellose sodium.

One Caucasian adult male living in Florida, USA spent several hours a day exercising outdoors in the direct sunlight each weekend, which often caused sunburn in exposed areas. This individual had previously discontinued use of conventional topical sunscreens some years earlier. After ingesting one capsule per day of the above preparation for five days, this individual was able to spend two full hours in the direct Florida sunlight for two days in a row without experiencing sunburn or skin inflammation. Throughout 30 days of continuous use, no sunburn or skin inflammation was experienced by this individual, despite regular direct sun exposure.

Thus it can be seen that ingestion of a photoprotectant composition of the invention several days prior to sun exposure can provide sunburn protection.

### EXAMPLE 5

Standard gelatin capsules were filled with 148 mg natural astaxanthin material from algae (Cyanotech Corporation), containing about 1.35% astaxanthin; 157 mg natural lycopene from tomatoes (LycoRed Corp.), containing 5% lycopene—5% average included for shelf life; and Polypodium (preferably Polypodium leucomotos) 30:1 extract 200 mg (Organic Hope Corporation), 50 mg stabilized superoxide dismutase (PL Thomas & Co. Inc.), 200 mcg selenomethionine (Pharmachem Corporation), along with excipients and carriers normally used in the capsule manufacturing process including vegetable stearates, silicon dioxide, and croscarmellose sodium.

One Caucasian adult female living in Florida, USA with a strong tendency for sunburn could not spend more than 15 minutes in the sun without experiencing sunburn and inflammation of the skin in exposed areas. After ingesting one capsule of the above preparation one hour prior to sun exposure, this individual spent a full hour in the direct Florida sun without experiencing skin inflammation or sunburn. Because this was an experiment to test the effectiveness of this formulation for this individual, she used no topical sunscreen during the exposure. Based on the positive results, this individual continued use of the above preparation daily for 60 days, and reported no sunburn or skin inflammation during that time, despite regular exposure to the Florida sun as part of her daily activities. When the 60-day supply of capsules ran out, she reported that her sun sensitivities and sunburns from brief sun exposures returned, and urgently requested additional capsules for personal use.

In this Example, ingestion immediately prior to sun exposure provided sun protective effects for both sunburn and skin inflammation, and discontinuance of oral ingestion of the formulation resulted in the return of sun sensitivity.

All patents, patent applications, and published references cited herein are hereby incorporated by reference in their entirety.

While this invention has been particularly illustrated and described with reference to particular examples, it will be understood by those skilled in the art that various changes in form and details may be made therein without departing from the scope and spirit of the invention encompassed by the appended claims.

1. A photoprotectant composition comprising at least one carotenoid and a Polypodium extract.

2. The composition of claim 1, wherein the at least one carotenoid comprises a carotenoid selected from the group consisting of astaxanthin, lycopene, canthaxanthin, β-carotene, lutein, and zeaxanthin.

3. The composition of claim 1, wherein the at least one carotenoid comprises astaxanthin.

4. The composition of claim 1, wherein the at least one carotenoid comprises lycopene.

5. The composition of claim 1, wherein the at least one carotenoid comprises astaxanthin and lycopene.

6. The composition of claim 1, wherein the Polypodium extract comprises an extract of a plant selected from the group consisting of Polypodium aureum, Polypodium crassifolium, Polypodium decumanum, Polypodium lanceolatum, Polypodium leucotomos, Polypodium percussum, Polypodium triseriale and Polypodium vulgare.

7. The composition of claim 6, wherein the polypodium extract comprises Polypodium leucotomos extract.

8.-9. (canceled)

10. A pharmaceutical composition comprising an effective photoprotectant amount of at least one carotenoid and a Polypodium extract, together with a pharmaceutically-acceptable carrier.

11. The pharmaceutical composition of claim 10, wherein the at least one carotenoid comprises a carotenoid selected from the group consisting of astaxanthin, canthaxanthin, β-carotene, lutein, and zeaxanthin.

12. The pharmaceutical composition of claim 10, wherein the at least one carotenoid comprises astaxanthin.

13. The pharmaceutical composition of claim 10, wherein the at least one carotenoid comprises lycopene.

14. The pharmaceutical composition of claim 10, wherein the at least one carotenoid comprises astaxanthin and lycopene.

15. The pharmaceutical composition of claim 10, wherein the polypodium extract comprises an extract of a plant selected from the group consisting of Polypodium aureum, Polypodium crassifolium, Polypodium decumanum, Polypodium lanceolatum, Polypodium leucotomos, Polypodium percussum, Polypodium triseriale and Polypodium vulgare.

16. The pharmaceutical composition of claim 15, wherein the polypodium extract comprises Polypodium leucotomos extract.

17. The pharmaceutical composition of claim 10, wherein the pharmaceutical formulation is formulated for oral administration.

18.-24. (canceled)

25. The pharmaceutical composition of claim 10, further comprising superoxide dismutase.

26. The pharmaceutical composition of claim 10, further comprising N-acetyl cysteine.

27. A packaged product comprising a container, a pharmaceutical composition comprising an effective photoprotectant amount of at least one carotenoid and a Polypodium extract, together with a pharmaceutically-acceptable carrier, and instructions for oral administration of the pharmaceutical composition to provide photoprotection.

28. A method of providing photoprotection to an individual, the method comprising administering to the individual a pharmaceutical composition comprising an effective photoprotectant amount of at least one carotenoid and a Polypodium extract.

29. The method of claim 28, wherein the at least one carotenoid comprises a carotenoid selected from the group consisting of astaxanthin, lycopene, canthaxanthin, β-carotene, lutein, and zeaxanthin.

30. (canceled)

31. The method of claim 28, wherein the at least one carotenoid comprises lycopene.

32. The method of claim 28, wherein the at least one carotenoid comprises astaxanthin and lycopene.

33.-39. (canceled)

40. The method of claim 28, wherein the step of administering comprises administering the pharmaceutical composition before, during or after sun exposure.

41. The method of claim 40, wherein the step of administering comprises administering the pharmaceutical composition two to three days before sun exposure.

42. A pharmaceutical composition in unit dose form, wherein the pharmaceutical composition comprises:
about 1 mg to about 6 mg astaxanthin;
about 1 mg to about 25 mg lycopene;
about 1 mg to about 300 mg Polypodium extract; and
a pharmaceutically acceptable carrier.
